(19) United States
(12) Patent Application Publication
(10) Pub. No.: US 2009/0075361 A1
(43) Pub. Date: Mar. 19, 2009

(54) Microfluidic Device and Method of Manufacturing the Microfluidic Device

(75) Inventors: Lisa DeLouise, Rochester, NY (US); Ut-Binh Thi Giang, South Rochester, NY (US); Michael King, Rochester, NY (US)

Correspondence Address: BOND, SCHOENECK & KING, PLLC, 10 BROWN ROAD, SUITE 201, ITHACA, NY, 14850-1248, US

(73) Assignee: University of Rochester, Rochester, NY (US)

(21) Appl. No.: 12/139,797

(22) Filed: Jun. 16, 2008

Related U.S. Application Data
(60) Provisional application No. 60/929,128, filed on Jun. 14, 2007.

(51) Int. Cl. C12M 1/34; B01J 19/00; G01N 21/01; B05D 3/00; C23C 16/513; C23C 14/24

(52) U.S. Cl. 435/287.9; 422/68.1; 422/82.05; 427/331; 427/578; 427/569; 427/294

(57) ABSTRACT

A microfluidic device having a substrate with an array of curvilinear cavities. The substrate of the microfluidic device is preferably fabricated of a polymer such as polydimethylsiloxane (PDMS). The microfluidic device is manufactured using a gas expansion molding (GEM) technique.

### REFERENCE TO RELATED APPLICATIONS

This application claims priority to U.S. Pat. No. 60/929,128, filed Jun. 14, 2007, which is hereby incorporated by reference.

### FIELD OF THE INVENTION

The present invention relates generally to microfluidics, and more particularly, to a microfluidic device and a method of manufacturing the microfluidic device.

### BACKGROUND

Microfluidics technology is used in microfluidic devices such as lab-on-a-chip systems (“LOC”), which separate or mix fluids and perform biochemical reactions using the separated or mixed fluids. Microfluidic devices include a substrate on which channels and chambers are formed. Soft lithography methods employing polymer-molding to generate LOC devices has enabled considerable innovation in the application of microfluidic devices for cell sorting and microcell culture. Precise control over laminar flow streams has enabled the selective spatial exposure of bioactive agents to cells and the investigation of mechanotransduction and cell response to shear stress under laminar or pulsitile flows. Many challenges exist however, to creating a technology platform that can sort cells from a diverse population, maintain them in culture, uniformly direct their fate (e.g., differentiation, elimination) and interrogate cell signaling responses in situ as a function of cell density.

Parallel-plate microchannel systems employing physical features (pillars) and adhesive interactions have proven useful in cell separation. The pillars are generally molded by a reverse molding technique. FIG. 1 is directed to a prior art method 10 used to fabricate pillar arrays. A silicon wafer 12 is provided. Deep reactive ion etching (DRIE) is used to make trenches 14 in silicon wafer 12. A hydrophobic material is coated onto silicon wafer mold 12 for easy removal of PDMS after it has been cured. PDMS 16 is cast onto the hydrophobic silicon wafer 12 as shown in Step (a). In Step (b), a vacuum is applied to degas PDMS 16 and deplete trapped gas, for example, air, nitrogen, helium or argon, in trenches 14 and dissolved in PDMS 16. PDMS 16 is then cured at 100° C. for two hours as shown in Step (c). The pillar array 18 is removed from silicon wafer mold 12.

FIG. 2 shows a pillar array for use in cell separation. The disadvantage of pillar arrays is that, over time, as the adherent cell number builds, the microchannel hydrodynamic resistance and flow velocity can change. Devices can become clogged.

It is a primary object of the invention to provide a microfluidic device that can integrate cell sorting, microcell culture and real time diagnostics. It is another object of the invention to provide a microfluidic device that is applicable for use in diagnostic, therapeutic and investigative research, particularly in the areas of stem cell and cancer biology. It is a further object of the invention to provide a facile and effective method of manufacturing microfluidic devices.

### SUMMARY OF THE INVENTION

These and other objects and advantages are accomplished by a microfluidic device. In one embodiment, the microfluidic device has a substrate with an array of curvilinear cavities. Curvilinear includes cavities that are bubbular or lacking a sharp edge or corner, such as spherical, oblong, oval or other curved shape. The cavities are provided in a variety of different arrays such as in evenly spaced rows or in staggered rows. The cavities may be all of the same size, all of the same shape, of varied sizes, and/or of varied shapes. The cavities may be spaced at a distance in a range of about two times the diameter of the opening of the cavities to about ten times the diameter of the opening of the cavities. The cavities may be fused cavities in the form of a linear tubular cavity.

In another embodiment, the substrate of the microfluidic device is preferably fabricated of a polymer such as polydimethylsiloxane (PDMS). Other examples include, but are not limited to, polysiloxanes, a carbon-based polymers, polyacrlyamides, polyacrylates, polymethacrylates or mixtures thereof.

In yet another embodiment, the cavities comprise a coating for selective capture of cells. The coating may provides a microenvironment beneficial for the culture of specific cells. The coating may comprise protein or biochemicals. The coating may be deposited by vacuum-assisted deposition. Examples of the coating include, but are not limited to, IgG, selectin, collagen, chemoattractant, signaling molecule, and/or fibronectin.

In yet another embodiment, the microfluidic device may have one or more sensors embedded therein. The sensors may be embedded in the substrate below the cavity or in the cavity, itself. An example of a sensor is an optical sensor.

In another embodiment, a method of manufacturing a microfluidic device using a gas expansion molding (GEM) technique is provided. A wafer mold is etched to provide one or more trenches therein. A polymer is applied onto the wafer mold, covering the one or more trenches to create an interface between the polymer layer and the wafer. The wafer with said polymer layer thereon sits for a period of time to allow gas, for example, air, nitrogen, helium, and/or argon, trapped in the one or more trenches to rise at the polymer-wafer interface. The gas may further combine with gas diffusing from the polymer, which mixture of gas rises to the interface. The polymer is cured, whereby the gas at the polymer-wafer interface expands to create microbubbles. Residual gas trapped in the polymer diffuses to enhance bubble growth. The cured polymer has cavities formed therein where the microbubbles had formed. The cavity shape depends on the mold parameters and process conditions. The polymer substrate having the cavities is separated from the wafer for use as a microfluidic reactor.

In a further embodiment, the step of allowing the wafer with the polymer layer thereon to sit is conducted for about 10 to about 60 minutes at a temperature in the range of about 20 to about 200° C. A preferred temperature range is from about 50 to about 100° C. One example of temperature in which this step occurs is room temperature.

In yet a further embodiment, the polymer is applied at a thickness in the range of about 0.1 to about 5000 microns. The polymer may be cured at a temperature in the range of about 20 to about 200° C.

In another embodiment, the wafer is coated with a hydrophobic material prior to the step of applying the polymer layer onto the wafer. Examples of hydrophobic materials include, but are not limited to, silane and fluorinated polymer coatings, such as Teflon. The coating may be produced by gas plasma deposition or chemical surface functionalization. The chemical surface functionalization may use alkoxy coupling agents comprising silanes, titanates, zirconates and zircoaluminates. Examples of silanes include, but are not limited to, 1H- or 2H-perfluoro-decyltrichlorosilane.

In yet a further embodiment, the trenches are provided in an array comprising trenches in evenly spaced rows, trenches in staggered rows, trenches of the same size, trenches of the same shape, trenches of varied sizes; and/or trenches of varied shapes. The shapes of the trenches include, but are not limited to, polygonal, circular, oval, and oblong cross-section. The polygonal cross-section may include, but is not limited to, triangular, square, rectangular, hexagonal or octagonal cross-section.

In a further embodiment, the trenches may have a depth in the range from about 10 microns to about 500 microns. A preferred depth is in the range of from about 25 microns to about 50 microns.

In a further embodiment, the trenches are positioned at a distance to create separate microbubbles or curvilinear cavities. The trenches may be spaced at a distance in the range of about two times the diameter of the opening of the trench to about ten times the diameter of the opening of the trench. The trenches may be spaced at a distance in the range of about 50 to about 500 microns.

In still another embodiment, the trenches may be positioned at a distance to create fused microbubbles or curvilinear cavities. The fused microbubbles or curvilinear cavities may create a single, linear tubular cavity.

In yet another embodiment, the cavities may be coated with a protein or with biochemicals for selective capture of cells. The coating may be applied by vacuum-assisted deposition. The coating may comprise, for example, IgG, selectin, collagen, chemoattractant, signaling molecule and/or fibronectin.

### BRIEF DESCRIPTION OF THE DRAWINGS

The present invention will be more fully understood and appreciated by reading the following Detailed Description in conjunction with the accompanying drawings, in which:

FIG. 1 is a schematic diagram showing manufacturing steps of a pillar substrate used in prior art microfluidic devices;

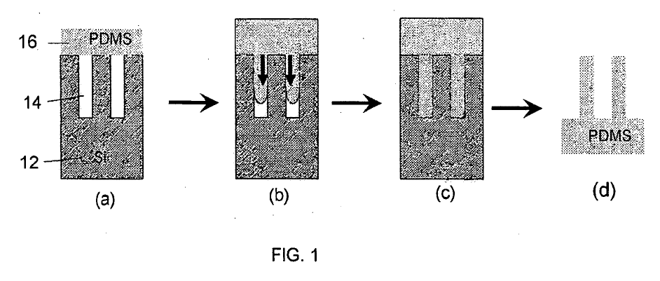

FIG. 2 is a micrograph of a reverse molded pillar array formed by conventional vacuum degassing techniques;

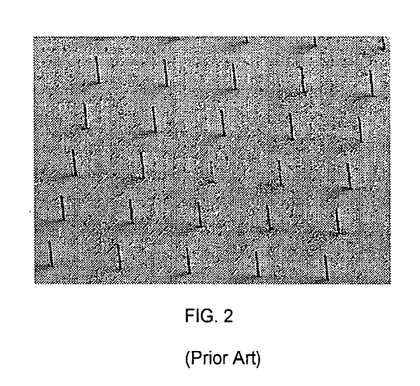

FIG. 3 is a schematic diagram of a substrate formed in accordance with the present invention;

FIG. 4 is a schematic diagram showing manufacturing steps of a substrate formed in accordance with the present invention;

FIG. 5 is a series of micrographs of microbubbles or microcavities formed during the GEM technique;

FIG. 6 is a micrograph of microbubbles formed during the GEM technique shown from an angular view;

FIG. 7 is a series of micrographs of various arrays of microbubbles formed in accordance with the present invention;

FIG. 8 is a micrograph of trenches having different shapes with microbubbles formed thereon;

FIG. 9 is a series of optical micrographs of mutant microbubbles formed using the GEM technique, illustrating the effect of trench opening and spatial arrangement thereof;

FIG. 10 is a diagram produced by finite element analysis of gas diffusion in a polymer structure containing preexisting bubbles;

FIG. 11 is a micrograph of a microbubble showing cells cultured in 6 days;

FIG. 12 is schematic diagram of a microbubble being coated using vacuum-assisted technology; and

FIG. 13 is a schematic diagram of a microfluidic device with an integrated microbubble device and an optical Porous Silicon (PSi) sensor.

### DETAILED DESCRIPTION

As will be appreciated, the embodiments of the present invention include a microfluidic device and a method of manufacture thereof. The microfluidic device is very useful for cell sorting, microcell culture and diagnostics in a single integrated device.

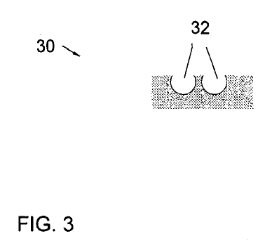

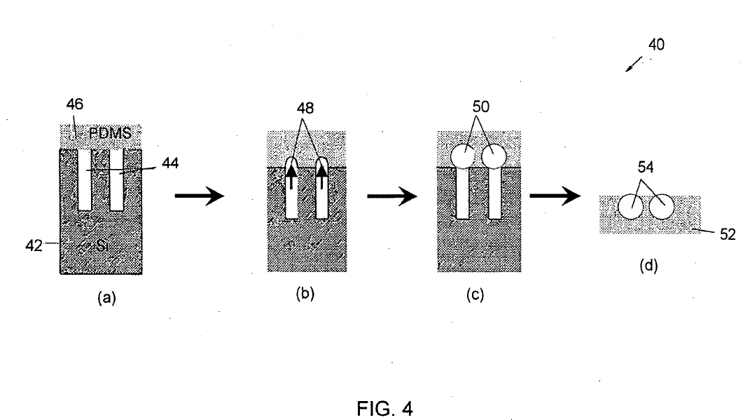

Reference is made to FIG. 3, which shows a substrate 30 formed in accordance with one embodiment of the invention, which substrate provides a microbubble array that is useful as a microfluidic device. Substrate 30 has two spherical cavities 32 formed therein. Substrate 32 is formed by a gas expansion molding (GEM) process 40 depicted in FIG. 4. A mask or mold 42 fabricated of Si or other similar material is manufactured by etching using the DRIE or similar process to produce trenches 44 therein. The trenches 44 may be any shape or form, including but not limited to square, triangular, circular and rectangular in cross-section. Trenches 44 may vary in diameter depending on the desired use of the end product, preferably in the range from about 20 to about 2000 microns, and more preferably in the range of about 60 to about 100 microns. By combining large (greater than 500 microns) with small (less than 200 microns), trench openings in composite devices can be formed consisting of reverse and bubble molded structures. Inconsistent GEM structures form trench openings less than about 60 microns using conventional polymeric materials, such as polymeric materials having a low modulus between 1.5 and 2.0 MPa. An example of one such material is polydimethylsiloxane (PDMS).

During the etching process, it is possible to produce a hydrophobic coating on the surface of the mold 42 through the cyclic reaction of the etchants used, for example, SF₆, followed by C₄F₈ passivation steps. Alternatively, a hydrophobic agent such as a silane reagent, (e.g., perfluorododecyl-1H-triethoxysilane) or plasma deposition of a Teflon-like coating may be used to produce a water surface contact angle in the range of from about 100 to about 150°. Depending on the treatment used, the mold may be immersed in a heptane solution for a period of time and/or immediately dried under a stream of N₂ gas, followed by heating to evaporate off the residual solvent. The use of a hydrophobic coating is important for the easy removal of the polymeric substrate in the final stage of the process.

In Step (a) of the process 40 shown in FIG. 4, a polymer 46 such as PDMS is cast over the hydrophobic mold and the sample is allowed to settle at room temperature for a period of time, for example, 30 minutes, to allow trapped gas bubbles to rise to the liquid/gas interface (Step b). Other polymeric material useful for substrates include, but are not limited to, inorganic polysiloxane (e.g., —Si—O—Si—O—) polymers, organic carbon based (e.g., —CH2—CHR—) polymers including, but not limited to, polyacrylamides, polyacrylates, and polymethacrylates.

A critical step in this process is the absence of a degassing step. No degassing is performed, as required in conventional reverse molding techniques. Polymer 46 is cured for a period of time, which time may vary depending on the polymer used. In this example, PDMS was used and curing occurred over a period of about two hours at about 100° C. This high temperature causes expansion of the gas in trenches 44. A meniscus 48 is formed over each trench 44 and serves to nucleate further bubble growth as trapped gas diffuses through the PDMS 46 to form microbubbles 50 (Step (c)). The cured PDMS 46 is removed from wafer mold 42 to reveal a substrate 52 having spherical cavities 54 formed therein as shown in Step (d).

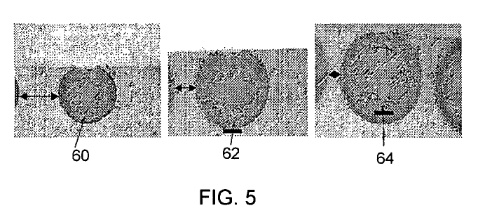

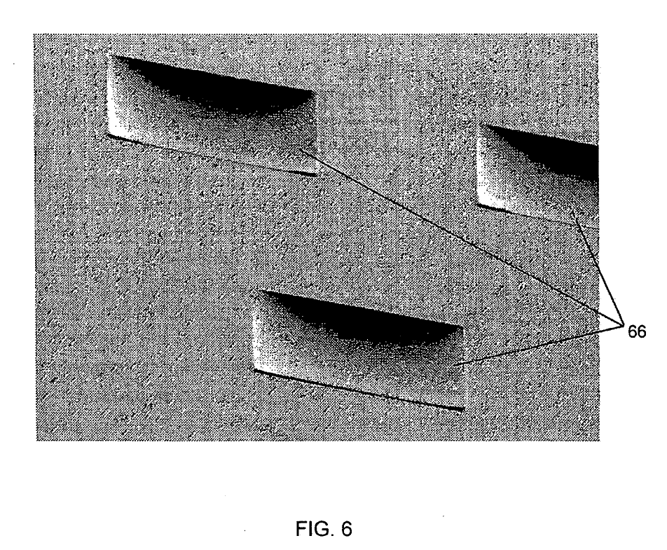

FIG. 5 shows variations in size of spherical cavities 60, 62 and 64 created from the microbubbles formed over a square trench opening during the curing process of the polymer. The final shape and size of the final cavities may depend on a variety of factors including, but not limited to, the thickness of the polymer layer poured over the wafer mold and the dimensions of the trenches in the wafer mold. The thicker the polymer layer applied onto the wafer mold, the bigger the bubble created, which is consistent with more residual gas. Keeping the depth of the trench constant, the size of the bubble increases as the area of the opening increases, i.e., the size of the bubble created is proportional to the size of the opening of the trench. FIG. 6 shows a series of curvilinear cavities 66 disposed in a substrate, viewed from an angled perspective.

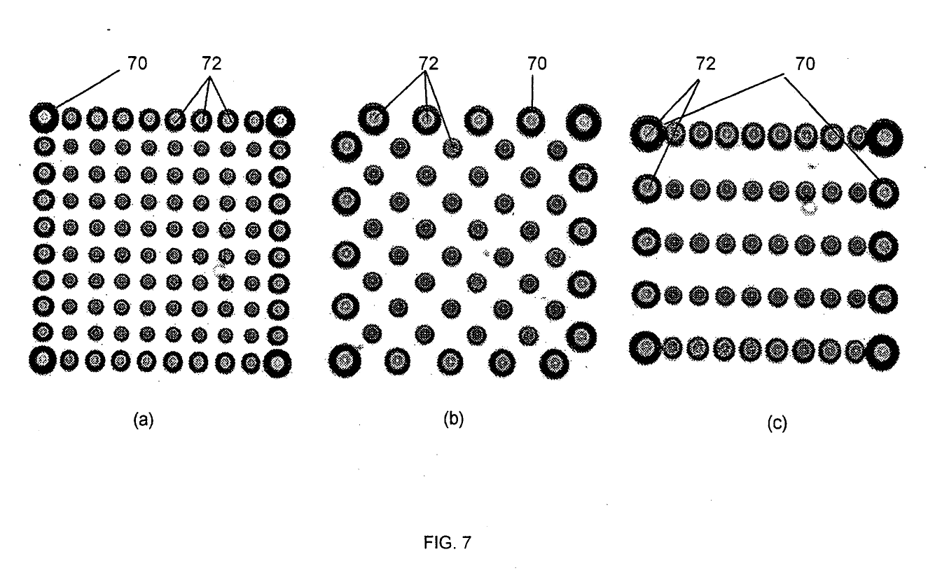

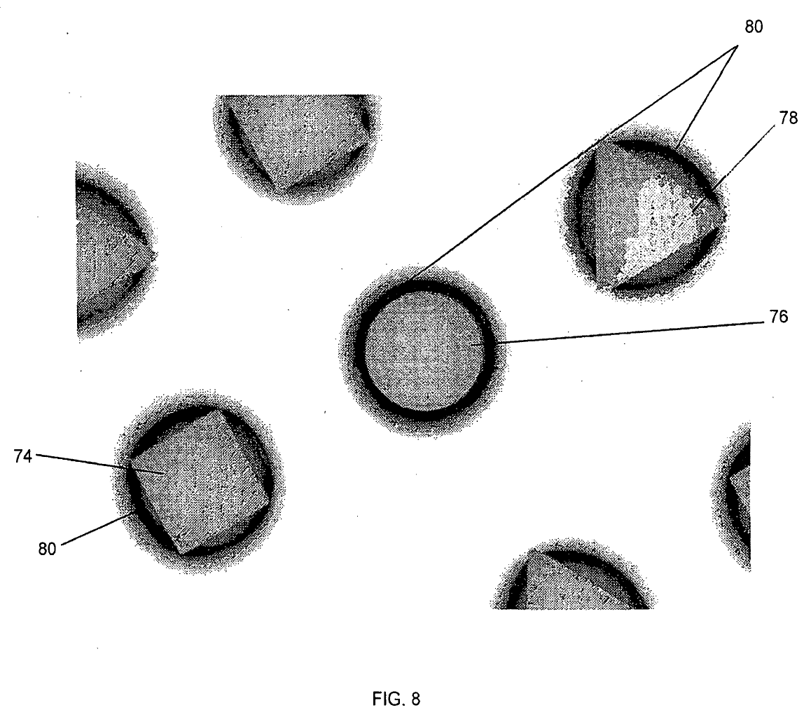

Spacing of the trenches, also known as mask openings, is another feature to consider when designing the spherical bubble array of this embodiment of the present invention. FIG. 7 depicts a variety of arrays that were produced from different arrays of mask openings. FIG. 7(a) depicts an array of microbubbles 70 formed over evenly spaced trenches 72. Trenches 72 all appear to be the same diameter, but microbubbles 70 differ in size depending on their location. Growth of microbubbles located at array corners is less constrained in FIGS. 7(a) and 7(c), and also along the outer rows in FIG. 7(b), which shows trenches in a staggered array. FIG. 7(c), showing alternating row array, in addition to the larger microbubbles located in the corners, also shows larger microbubbles in the end rows running longitudinally. The end rows running laterally, are slightly larger than the microbubbles located interiorly, but smaller than the corners and end rows running longitudinally. While not wishing to be bound by any theory, it is thought that this phenomenon is related to the depletion of trapped gas in the polymer premix that can diffuse to the expanding bubble. FIG. 8 shows various shaped mask openings 74 (square), 76 (round), and 78 (triangular) having microbubbles 80 formed thereon, all having the same spherical shape.

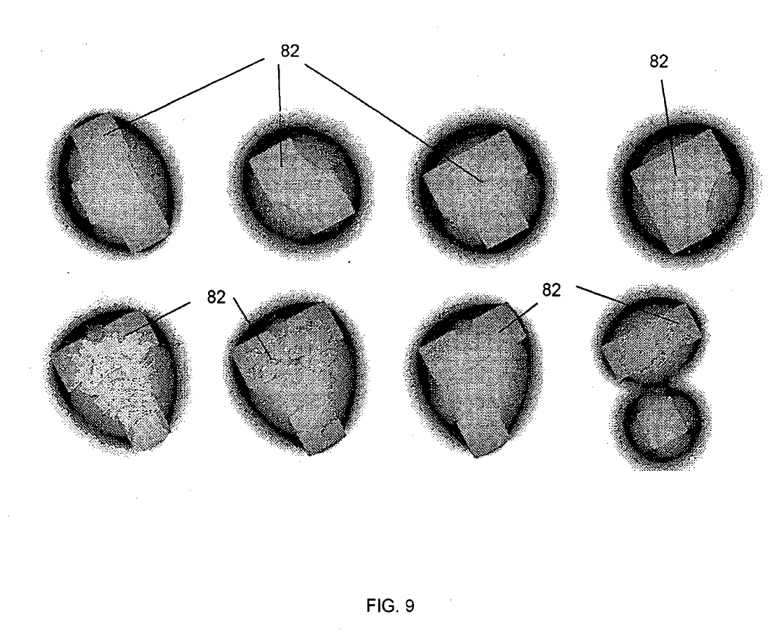

Trench openings that are spaced close to each other, such as less than one diameter apart, may produce mutant bubbles 82, as shown in FIG. 9. During the polymer cure process, expanding bubbles can fuse forming obtuse shapes depending upon the spatial alignment of trench openings. It is also possible that the polymer film between adjacent microbubble openings is torn off during the mold separation process creating a much larger microbubble opening. Mutant bubbles exhibit unique flow properties. One example of the formation of mutant bubbles has been found using trench openings greater than about 40 microns in diameter with polymer thicknesses of about 0.5 to about 5 mm.

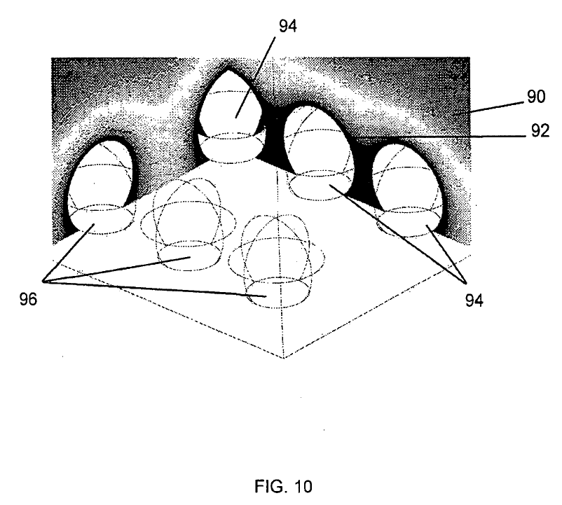

FIG. 10 is directed to a micrograph created using finite element analysis. As noted above, it has been determined that close packed trenches produce smaller microbubbles whereas isolated trenches (e.g., inter-trench spacing greater than about 10× the trench diameter) produce larger bubbles. While not wishing to be bound by any theory, it is thought that rapid expansion of gas in the trench nucleates a meniscus and bubble growth recruits trapped gas in the polymer. Competition for gas in the polymer is a limiting factor in bubble growth during the cure process.

In FIG. 10, preliminary simulations were conducted to determine if the problem of vapor bubble growth is tractable. The initial conditions positioned pre-existing bubbles in a polymer containing dissolved gas above a trench. Bubbles were treated as sinks. Calculations were carried out with respect to the change in gas concentration as a function of time using arbitrary units. Gas concentration is indicated on a color scale with red 90 being high and blue 92 being none. Bubbles 94 on the right side of the plot are spaced more closely than the bubbles 96 on the left axis, which are spaced farther apart. After a fixed diffusion time, gas dissolved in the polymer is fully depleted between the closely spaced features 94, suggesting bubble growth would be limited, whereas gas is still available between the larger spaced bubbles 96. Factors that affect the microbubble formation include, but are not limited to, polymer thickness, gas concentration, cure temperature, ramp rate and trench depth.

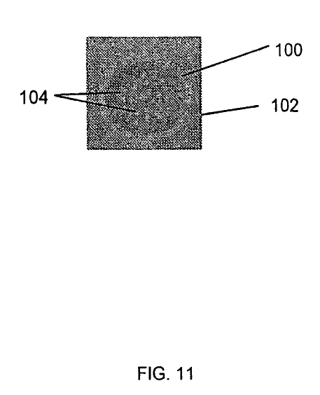

In a method of using the microbubble arrays, fluid flow properties are examined. An example of a use of the arrays is for the deposition of cancer cells using flow or gravity to sustain these cells in a microculture. FIG. 11 shows a spherical cavity 100 made in accordance with this embodiment of the invention disposed in a substrate 102 having cultured cells 104 therein.

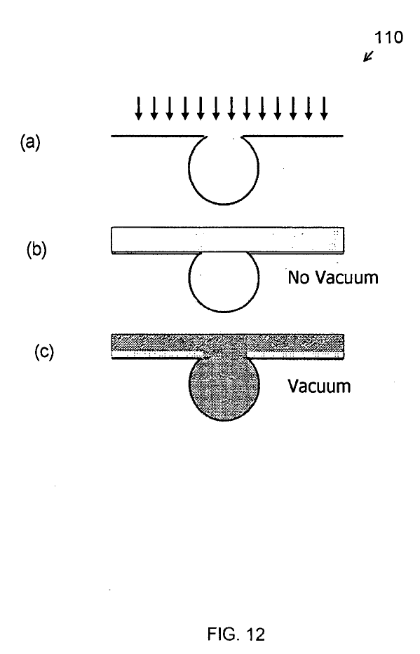

A key requirement for utilizing microbubble arrays in cell sorting and microcell culture, examples of two forms of use of the microbubble array herein, is the ability to selectively and spatially alter the polymer surface chemistry. FIG. 12 is a schematic diagram of a vacuum-assisted coating (VAC) procedure 110 used to selectively coat bioactive molecules onto a microbubble wall 112 and the microchannel surface 114. The capability is desirable as it may be advantageous to coat the microchannel surface 114 with a blocking agent, such as bovine serum albumin (BSA) or polyethylene glycol (PEG) to prevent nonspecific cell interactions while depositing a chemotactic (e.g., stromal-derived factor-1 (SDF-1), IL-8) or extracellular matrix molecule, such as a protein (e.g., collagen, fibronectin), or an adhesive (e.g., selectin) in the microbubble wells 112 to enhance cell capture, adhesion or bioactive molecule to direct cell fate. The VAC process takes advantage of the intrinsic PDMS polymer hydrophobicity (θ˜105°).

As an example, the VAC process may begin with Step (a), where the polymer sample is exposed to UV-ozone (BioForce Nanosciences ProCleaner) for 1 hour. Although used in this non-limiting example, the VAC process may be conducted without the use of this ozone step. Alternative methods may also be used to render the surface hydrophilic including, but not limited to, the application of oxygen gas plasma. This treatment makes the ozone-contacted surfaces hydrophilic (θ<105°). A shadowing effect occurs that renders the undersurface at the microbubble entrance 112 sufficiently hydrophobic to inhibit aqueous solutions, gently dispensed on the planar surface 114, from entering the microbubble 112, as shown in Step (b). The application of a vacuum in Step (c) indicates that it is possible to coat bioactive molecules on the microchannel surface 114 that may or may not differ from those deposited in the well 112.

Microfluidic flows using microparticle image velocimetery of isolated microbubbles (nearest microbubble is greater than about 10× bubble diameter opening) with square (80 microns) opening reveal asymmetric fluid flows above the entrance that extend into the cavity. Asymmetric flows depend on microbubble size and fluid shear stress. Under a constant shear stress of 5 dyn/cm² (about 0.2 ml/min) microparticles enter the cavity at the down stream edge. Stable asymmetric recirculating vortices develop causing the microparticles to exhibit a triangular velocity trace in the cavity with fore-aft asymmetry. Along the microbubble well bottom they flow counter to the main stream. At the top of the cavity, they flow parallel to the main stream.

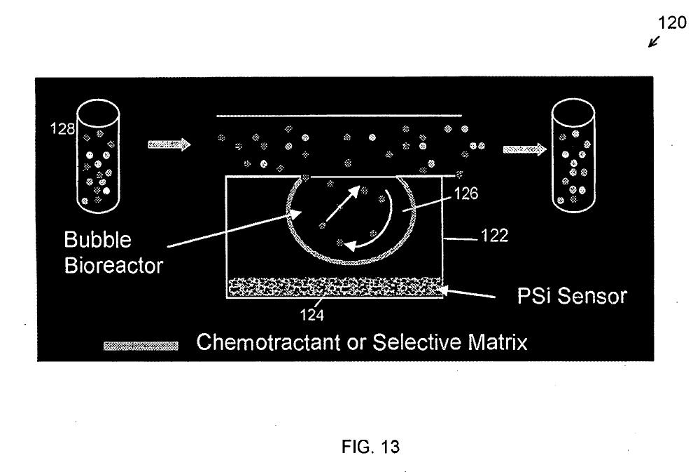

FIG. 13 is just one example of a device 120 that incorporates the microbubble array of the embodiments of the present invention. A substrate 122 includes an optical biosensor 124 such as Porous Silicon (PSi) embedded therein. A spherical cavity 126 is disposed in substrate 122. A chemotractant causes target cells to come form the cell mixture 128 into the spherical cavity 126. The PSi optical sensor 124 is designed to detect certain types of cells collected in spherical cavity 126, or to monitor specific chemical signals (cytokines, gases, small molecules) or cell concentrations within spherical cavity 126 in real time.

As described herein, a microfluidic device is provided using GEM molding and VAC techniques. The microbubble features are applicable to diagnostic, therapeutic, and investigative purposes in the many research areas including stem and cancer cell research. Examples of devices that can incorporate the microbubble feature include, but are not limited to, devices for drug screening, pharmacokinetic analysis, cytotoxicity analysis, cell capture, quorum sensing, isolation of cells, and chemotherapeutic response of cells.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended embodiments.

What is claimed is:

1. A microfluidic device comprising:
a substrate having one or more curvilinear cavities.

2. The microfluidic device of claim 1 wherein the curvilinear cavities are spherical, oblong, or oval in shape.

3. The microfluidic device of claim 1 wherein the substrate is fabricated of a polymer.

4. The microfluidic device of claim 3 wherein the polymer comprises a polysiloxane, a carbon-based polymer or mixtures thereof.

5. The microfluidic device of claim 3 wherein the polymer comprises PDMS, a polyacrlyamide, a polyacrylate, a polymethacrylate or a mixture thereof.

6. The microfluidic device of claim 1 wherein the curvilinear cavities are provided in an array comprising cavities arranged in evenly spaced rows, cavities in staggered rows, cavities of the same size, cavities of the same shape, cavities of varied sizes; and/or cavities of varied shapes.

7. The microfluidic device of claim 6 wherein the cavities are spaced at a distance in a range of about two times the diameter of the opening of the cavities to about ten times the diameter of the opening of the cavities.

8. The microfluidic device of claim 1 wherein the cavities comprise fused cavities in the form of a linear tubular cavity.

9. The microfluidic device of claim 1 wherein the cavities comprise a coating for selective capture of cells.

10. The microfluidic device of claim 9 wherein the coating provides a microenvironment beneficial for the culture of specific cells.

11. The microfluidic device of claim 9 wherein the coating comprises protein or biochemicals.

12. The microfluidic device of claim 9 wherein the coating is deposited by vacuum-assisted deposition.

13. The microfluidic device of claim 9 wherein the coating comprises IgG, selectin, collagen chemoattractant, signaling molecule, and/or fibronectin.

14. The microfluidic device of claim 1 wherein the substrate further comprises one or more sensors embedded therein.

15. The microfluidic device of claim 14 wherein the sensors comprise optical sensors.

16. The microfluidic device of claim 1 further comprising sensors disposed in the curvilinear cavities.

17. The microfluidic device of claim 16 wherein the sensors comprise optical sensors.

18. A method of manufacturing a microfluidic device comprising:
providing a wafer having one or more trenches therein;
applying a polymer layer onto the wafer, covering the one or more trenches to create an interface between the polymer layer and the wafer;
allowing the wafer with said polymer layer thereon to sit to allow gas trapped in the one or more trenches to rise at the polymer-wafer interface;
curing the polymer, whereby the gas at the polymer-wafer interface expands to create microbubbles;
separating the polymer from the wafer to provide a polymer substrate having one or more curvilinear cavities therein.

19. The method of claim 18 wherein the step of allowing the wafer with said polymer layer thereon to sit to allow gas trapped in the one or more trenches to rise at the polymer-wafer interface further comprises allowing gas trapped in the one or more trenches to combine with gas diffusing from the polymer.

20. The method of claim 18 the curvilinear cavities are spherical, oblong, or oval in shape.

21. The method of claim 18 wherein the step of allowing the wafer with said polymer layer thereon to sit is conducted for about 10 to about 60 minutes at a temperature in the range of about 23 to about 200° C.

22. The method of claim 21 wherein the temperature is in the range of about 50 to about 100° C.

23. The method of claim 18 wherein the step of allowing the wafer with said polymer layer thereon to sit is conducted at room temperature.

24. The method of claim 18 wherein the polymer comprises a polysiloxane, a carbon-based polymer or mixtures thereof.

25. The method of claim 18 wherein the polymer comprises PDMS, a polyacrlyamide, a polyacrylate, a polymethacrylate or a mixture thereof.

26. The method of claim 18 wherein the polymer is applied at a thickness in the range of about 0.1 to about 5000 microns.

27. The method of claim 18 wherein the polymer is cured at a temperature in the range of about 23 to about 200° C.

28. The method of claim 18 further comprising coating the wafer with a hydrophobic material prior to the step of applying the polymer layer onto the wafer.

29. The method of claim 28 wherein the hydrophobic material comprises silane or fluoronated polymer coating.

30. The method of claim 30 wherein the hydrophobic material comprises a coating produced by gas plasma deposition or chemical surface functionalization.

31. The method of claim 30 wherein the chemical surface functionalization uses alkoxy coupling agents comprising silanes, titanates, zirconates and zircoaluminates.

32. The method of claim 31 wherein silane comprises 1H- or 2H-perfluoro-decyltrichlorosilane.

33. The method of claim 18 wherein the trenches are provided in an array comprising trenches in evenly spaced rows, trenches in staggered rows, trenches of the same size, trenches of the same shape, trenches of varied sizes; and/or trenches of varied shapes.

34. The method of claim 33 wherein the shape of the trenches comprise polygonal, circular, oval, or oblong cross-section.

35. The method of claim 34 wherein the polygonal cross-section comprises triangular, square, rectangular, hexagonal or octagonal cross-section.

36. The method of claim 18 wherein the trenches have a depth in the range of from about 10 microns to about 500 microns.

37. The method of claim 35 wherein the depth is in the range of from about 25 microns to about 50 microns.

38. The method of claim 18 wherein the trenches are positioned at a distance to create separate microbubbles.

39. The method of claim 38 wherein the separate microbubbles create separate spherical cavities.

40. The method of claim 38 wherein the trenches are spaced at a distance in the range of about two times the diameter of the opening of the trench to about ten times the diameter of the opening of the trench.

41. The method of claim 38 wherein the trenches are spaced at a distance in the range of about 50 microns to about 500 microns.

42. The method of claim 18 wherein the trenches are positioned at a distance to create fused microbubbles.

43. The method of claim 42 wherein the fused microbubbles create a single, linear tubular cavity.

44. The method of claim 42 wherein the trenches are spaced at a distance in the range of about two times the diameter of the opening of the trench to about ten times the diameter of the opening of the trench.

45. The method of claim 42 wherein the trenches are spaced at a distance in the range of about 50 microns to about 500 microns.

46. The method of claim 18 further comprising coating the spherical cavities in the polymer substrate with a protein or biochemicals for selective capture of cells.

47. The method of claim 46 wherein the coating is deposited by vacuum-assisted deposition.

48. The method of claim 46 wherein the coating comprises IgG, selectin, collagen chemoattractant, signaling molecule and/or fibronectin.
